(19) United States
(12) Patent Application Publication
(10) Pub. No.: US 2020/0322454 A1
(43) Pub. Date: Oct. 8, 2020

(54) WIRELESS ULTRASOUND DEVICE AND RELATED APPARATUS AND METHODS

(71) Applicant: Butterfly Network, Inc., Guilford, CT (US)

(72) Inventors: Krishna Ersson, Guilford, CT (US); Lawrence Spector, Guilford, CT (US); Hamid Soleimani, Guilford, CT (US); Graham Peyton, Madison, CT (US)

(21) Appl. No.: 16/838,883

(22) Filed: Apr. 2, 2020

Related U.S. Application Data
(60) Provisional application No. 62/829,980, filed on Apr. 5, 2019.

(51) Int. Cl. H04L 29/06; H04L 29/08; G06F 9/54; G16H 40/67; G16H 30/20; A61B 8/00

(52) U.S. Cl. CPC H04L 67/40; H04L 67/1095; G06F 9/547; H04W 4/80; G16H 30/20; A61B 8/565; G16H 40/67

(57) ABSTRACT

A system comprising an ultrasound device configured to communicate ultrasound data wirelessly to a remote computing device over a wireless communication link, wherein the ultrasound device and the remote computing device implement the wireless communication link using a set of functions to wirelessly transmit the ultrasound data.

### CROSS-REFERENCE TO RELATED APPLICATIONS

This application claims the benefit under 35 U.S.C. § 119(e) of U.S. Provisional Application Ser. No. 62/829,980, entitled “WIRELESS ULTRASOUND DEVICE AND RELATED APPARATUS AND METHODS” filed on Apr. 5, 2019, under Attorney Docket Number B1348.70137US00, which is incorporated herein by reference in its entirety.

### FIELD

The present application relates to computerized techniques for providing wireless communication of ultrasound data, and in particular to techniques for wirelessly transmitting data of high-resolution images of a subject from a wireless ultrasound device.

### BACKGROUND

Ultrasound imaging systems typically include an ultrasound probe connected to a host by a cable, such as an analog cable. The ultrasound probe is controlled by the host to emit and receive ultrasound signals. The received ultrasound signals are processed to generate an ultrasound image.

### SUMMARY

Aspects of the present application relate to computerized techniques for providing wireless communication functionality to an ultrasound device.

Some embodiments are directed to a method including implementing a wireless communication link for ultrasound data using a set of functions to wirelessly transmit the ultrasound data. In some embodiments, one or more functions of the set of functions can be executed using a remote procedure call (RPC) protocol. In some embodiments, the method includes receiving, from an endpoint of the wireless communication link, a request to execute a function from the set of functions.

In some embodiments, the method includes executing a function associated with the request to execute the function from the set of functions. In some embodiments, a function from the set of functions comprises an asynchronous function. In some embodiments, a function from the set of functions comprises a synchronous function. In some embodiments, the method includes transmitting a response to the endpoint. In some embodiments, a function from the set of functions comprises a reset function. In some embodiments, a function from the set of functions comprises a send function. In some embodiments, a function from the set of functions comprises a receive function. In some embodiments, a function from the set of functions comprises a start function. In some embodiments, a function from the set of functions comprises a stop function.

In some embodiments, the wireless communication link includes a set of channels. In some embodiments, the set of channels comprises a transmit channel and a receive channel. In some embodiments, the method includes receiving ultrasound data from a wired link, and transmitting, via the wireless communication link, at least a portion of the ultrasound data to an endpoint of the wireless communication link.

In some embodiments, the wired link comprises a set of channels, and transmitting includes transmitting data from a first channel of the set of channels of the wired link over a second channel of the set of channels of the wireless communication link. In some embodiments, the first channel is different than the second channel.

In some embodiments, the method includes receiving data from the wireless communications link, and transmitting, via a second link, at least a portion of the data to an endpoint of the second link. In some embodiments, the second link includes a set of channels, and transmitting includes transmitting data from a first channel of the set of channels of the wireless communication link over a second channel of the set of channels of the second link.

In some embodiments, the ultrasound data is sufficient for forming one or more ultrasound images therefrom. In some embodiments, the one or more ultrasound images include information indicative of a size or shape of a tumor. In some embodiments, the one or more ultrasound images include information indicative of an elasticity of a tumor. In some embodiments, the one or more ultrasound images include information indicative of a vascularity of a tumor. In some embodiments, the one or more ultrasound images include information indicative of a fullness or a volume of a bladder. In some embodiments, the one or more ultrasound images include information indicative of a bladder outlet obstruction. In some embodiments, the one or more ultrasound images include information indicative of a pulse wave velocity or a blood pressure.

In some embodiments, the one or more ultrasound images include information indicative of a diameter of a blood vessel. In some embodiments, the one or more ultrasound images include information indicative of a volume of a blood vessel. In some embodiments, the one or more ultrasound images include information indicative of a collapsibility of a blood vessel. In some embodiments, the one or more ultrasound images include information indicative of a blood flow rate through a blood vessel. In some embodiments, the one or more ultrasound images include information indicative of a wall motion of a blood vessel. In some embodiments, the blood vessel is an inferior vena cava. In some embodiments, the one or more ultrasound images include information indicative of an intravascular volume. In some embodiments, the one or more ultrasound images include information indicative of a left ventricular wall motion. In some embodiments, the one or more ultrasound images include information indicative of a cardiac output.

In some embodiments, the one or more ultrasound images include information indicative of an ejection fraction. In some embodiments, the one or more ultrasound images include information indicative of a central venous pressure. In some embodiments, the one or more ultrasound images include information indicative of a peripheral blood flow rate. In some embodiments, the one or more ultrasound images include information indicative of an abdominal aortic aneurysm. In some embodiments, the one or more ultrasound images include information indicative of a deep vein thrombosis. In some embodiments, the one or more ultrasound images include information indicative of a fetal heartbeat. In some embodiments, the one or more ultrasound images include information indicative of a pregnancy.

In some embodiments, the one or more ultrasound images include information indicative of a follicle growth in ovaries. In some embodiments, the one or more ultrasound images include information indicative of dehydration. In some embodiments, the one or more ultrasound images include information indicative of a skin moisturization. In some embodiments, the one or more ultrasound images include information indicative of an amniotic fluid volume. In some embodiments, the one or more ultrasound images include information indicative of pneumonia. In some embodiments, the one or more ultrasound images include information indicative of a pleural effusion.

In some embodiments, the one or more ultrasound images include information indicative of pneumothorax. In some embodiments, the one or more ultrasound images include information indicative of chronic obstructive pulmonary disease (COPD). In some embodiments, the one or more ultrasound images include information indicative of a lung volume. In some embodiments, the one or more ultrasound images include information indicative of an enteral feeding tube placement. In some embodiments, the one or more ultrasound images include information indicative of a gastric emptying. In some embodiments, the one or more ultrasound images include information indicative of indigestion.

In some embodiments, the one or more ultrasound images include information indicative of a kidney function. In some embodiments, the one or more ultrasound images include information indicative of a renal blood flow rate. In some embodiments, the one or more ultrasound images include information indicative of a needle placement during pericardiocentesis. In some embodiments, the one or more ultrasound images include information indicative of a needle placement during amniocentesis. In some embodiments, the one or more ultrasound images include information indicative of a vasospasm. In some embodiments, the one or more ultrasound images include information indicative of a high-intensity focused ultrasound ablation. In some embodiments, the one or more ultrasound images include information indicative of a drug delivery. In some embodiments, the one or more ultrasound images include information indicative of a hemodialysis access.

In some embodiments, the method includes wirelessly offloading the ultrasound data using the wireless communication link at a data rate within a range of approximately 140-180 megabits/second. In some embodiments, implementing the wireless communication link comprises implementing the communication link using an IEEE 802.11 standard.

In some embodiments, the method includes wirelessly offloading the ultrasound data from an ultrasound device. In some embodiments, the ultrasound device includes a handheld ultrasound probe. In some embodiments, the ultrasound device includes a wearable ultrasound device. In some embodiments, the wearable ultrasound device comprises an ultrasound patch. In some embodiments, the ultrasound device comprises an ultrasound pill.

Some embodiments are directed to a method including converting ultrasound data from a first format for wired communication to a second format for wireless communication to generate converted ultrasound data. The method can include wirelessly offloading, from an ultrasound device, the converted ultrasound data. The method can include wirelessly offloading, from a second device physically connected to an ultrasound device, the converted ultrasound data.

In some embodiments, the ultrasound device comprises a handheld ultrasound probe. In some embodiments, the ultrasound device comprises a wearable ultrasound device. In some embodiments, the wearable ultrasound device comprises an ultrasound patch. In some embodiments, the ultrasound device comprises an ultrasound pill.

In some embodiments, wirelessly offloading the converted ultrasound data includes wirelessly offloading the converted ultrasound data at a data rate within a range of approximately 140-180 megabits/second. In some embodiments, wirelessly offloading the converted ultrasound data includes wirelessly offloading the converted ultrasound data using a communication platform employing an IEEE 802.11 standard. In some embodiments, the first format is a format for a USB link. In some embodiments, the second format is a format for a wireless link.

### BRIEF DESCRIPTION OF THE DRAWINGS

Various aspects and embodiments of the disclosed technology will be described with reference to the following figures. It should be appreciated that the figures are not necessarily drawn to scale. Items appearing in multiple figures are indicated by the same reference number in all the figures in which they appear.

FIG. 1A is a diagram illustrating a first exemplary embodiment of a handheld probe comprising an ultrasound probe with built-in wireless communication, in accordance with some embodiments of the technology described herein.

FIG. 1B is a diagram illustrating a second exemplary embodiment of a handheld probe comprising an ultrasound probe with a connected wireless communication module, in accordance with some embodiments of the technology described herein.

FIG. 2 illustrates an exemplary embodiment of a patch comprising an ultrasound probe and built-in wireless communication, in accordance with some embodiments of the technology described herein.

FIG. 3 illustrates a pill comprising an ultrasound probe and built-in wireless communication, in accordance with some embodiments of the technology described herein.

FIG. 4 is a diagram illustrating how an ultrasound device may be used to image a subject, in accordance with some embodiments of the technology described herein.

FIG. 5 illustrates a wireless architecture with two endpoints, in accordance with some embodiments of the technology described herein.

FIG. 6 illustrates a wireless architecture with two endpoints and multiple channels, in accordance with some embodiments of the technology described herein.

FIG. 7 illustrates a wireless architecture with four endpoints, in accordance with some embodiments of the technology described herein.

FIG. 8 is a flowchart of an illustrative process for operating a wireless communication link, in accordance with some embodiments of the technology described herein.

FIG. 9 is a flowchart of an illustrative process of converting data from one communication link for transmission over a second wireless communication link, in accordance with some embodiments of the technology described herein.

### DETAILED DESCRIPTION

The present disclosure describes aspects of an ultrasound device configured to image a subject and wirelessly transmit the image data to a remote device. The wireless communication links, as well as wired communication links, that are available to transmit data between the ultrasound device and a remote device can be implemented using classes to abstract the underlying hardware and/or software implementations into one or more common interfaces. A wireless communication link can be incorporated into an existing communication link configuration (e.g., a physical communication link) between the ultrasound device and the remote device without requiring changes to the ultrasound device and/or the remote device by leveraging remote function calls across the wireless interface to the existing communication link interfaces. As a result, wireless communication functionality can be incorporated into an ultrasound device (such as an ultrasound probe, patch or pill, as described herein) without requiring modifications to aspects of the ultrasound device and/or remote device.

Some embodiments are directed to implementing a wireless communication link for transmitting ultrasound data using a set of functions to wirelessly transmit the ultrasound data. In some embodiments, the functions can be executed using remote function calls, such as using a remote procedure call (RPC) protocol. In some embodiments, the wireless communication link is added between an existing set of one or more communication links between an ultrasound device and a remote device. A function called by and endpoint (e.g., the ultrasound device or remote device) and/or other data transmitted by the endpoint can be passed through the wireless communication link using the remote function calls. The receiving endpoint can receive the passed functions and/or data as if the endpoints were connected using just the existing set of one or more communication links.

The inventors have recognized that ultrasound probes are often limited because they require a physical connection to transmit the ultrasound image data to a receiving device (e.g., to a computer, mobile phone, and/or other computing device). For example, a physical cable such as a USB cable is often used to connect an ultrasound probe with the receiving device. As a result, a medical professional typically needs to have both the ultrasound probe and receiving device in the same room. The cable can be cumbersome because it often limits the medical professional's range of movement and potentially interferes with other items in the room (e.g., such as a hospital bed, tables, other people, etc.).

By contrast, the ultrasound device developed by the inventors and described herein, is configured to wirelessly transmit ultrasound imaging data of a sufficiently high resolution for forming medically-relevant images. A wireless communication link can be added to an existing communication configuration using one or more wireless managers to facilitate the wireless communication link. In some embodiments, a wireless manager is used to invoke remote function calls to transmit data from an endpoint (e.g., ultrasound device or remote device) over the wireless link to the other endpoint. For example, the ultrasound device may be configured to use a first communication link (e.g., a wired link, such as USB, Ethernet, serial, and/or the like) to communicate with the remote device. The wireless link can be added in-between the existing first communication link, such that the endpoints still communicate as if only the first communication link is between the endpoints. A wireless manager can be incorporated on each side of the wireless link to pass functions and/or other data from each endpoint over the wireless communication link (e.g., by invoking the necessary remote function calls to pass functions called by an endpoint for the first communication link over the wireless communication link).

An ultrasound device may be implemented in any of a variety of physical configurations including, for example, as a part of an internal imaging device, such as a pill to be swallowed by a subject or a pill mounted on an end of a scope or catheter, as part of a handheld probe, as part of a patch configured to be affixed to the subject, and/or the like. The wireless communication techniques described herein can be used for any of such various configurations, as described further herein. In some embodiments, the ultrasound devices described herein can leverage the hardware architectures developed by the assignee to provide wireless communication functionality for ultrasound devices.

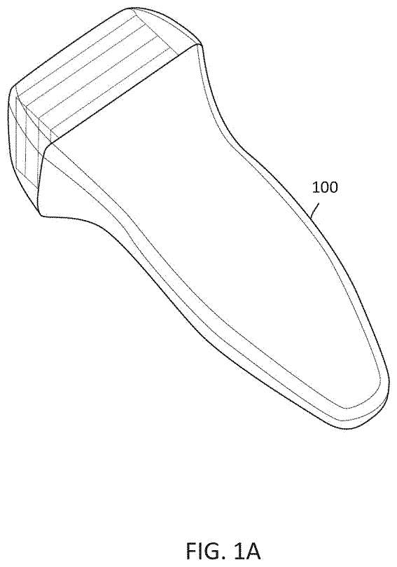

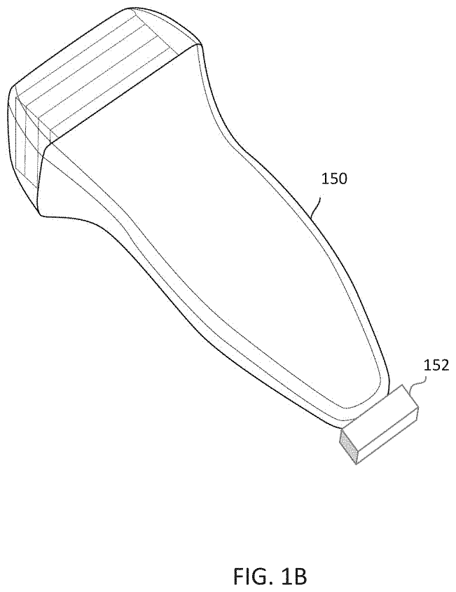

FIG. 1A is a diagram illustrating a first exemplary embodiment handheld probe 100 comprising an ultrasound probe with built-in wireless communication, in accordance with some embodiments of the technology described herein. The hand-held probe 100 is configured to transmit ultrasound data collected by the probe 100 wirelessly to one or more external host devices (not shown in FIG. 1A) for further processing, using the techniques for wireless communication of ultrasound data described herein.

In some embodiments, the handheld probe 100 can include one or more circuits (e.g., field programmable gate arrays (FPGAs), application specific integrated circuits (ASICs), processors, central processing units (CPUs), an ultrasound-on-a-chip, and/or the like) to perform the ultrasound imaging and to process the ultrasound image data. The probe 100 may be used in any of a number of imaging and/or treatment applications. In one illustrative implementation, for example, an imaging device including an N×M planar or substantially planar array of elements may be used to acquire an ultrasonic image of a subject, e.g., a person's abdomen, by energizing some or all of the elements in the array(s) (either together or individually) during one or more transmit phases, and receiving and processing signals generated by some or all of the elements in the array(s) during one or more receive phases, such that during each receive phase the elements sense acoustic signals reflected by the subject. In other implementations, some of the elements in the array(s) may be used only to transmit acoustic signals and other elements in the same array(s) may be simultaneously used only to receive acoustic signals.

The handheld probe 100 can also include one or more additional circuits (e.g., FPGAs, ASICSs, CPUs, wireless communication circuits, antennas, etc.) that are used to offload the ultrasound data from the ultrasound probe. In some embodiments, the handheld probe can be configured to wirelessly transmit data as described in U.S. Provisional Patent Application No. 62/655,158, titled “Methods and Apparatuses for Offloading Ultrasound Data,” assigned to the assignee of the present application, which is hereby incorporated by reference herein in its entirety.

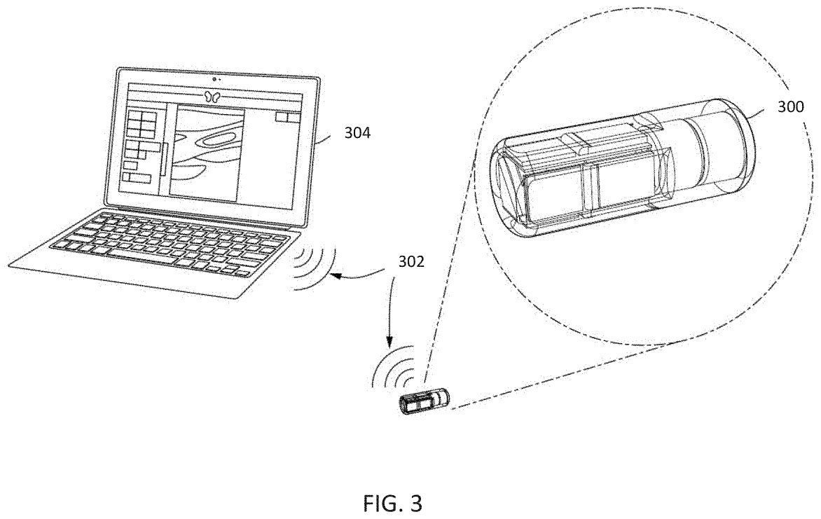

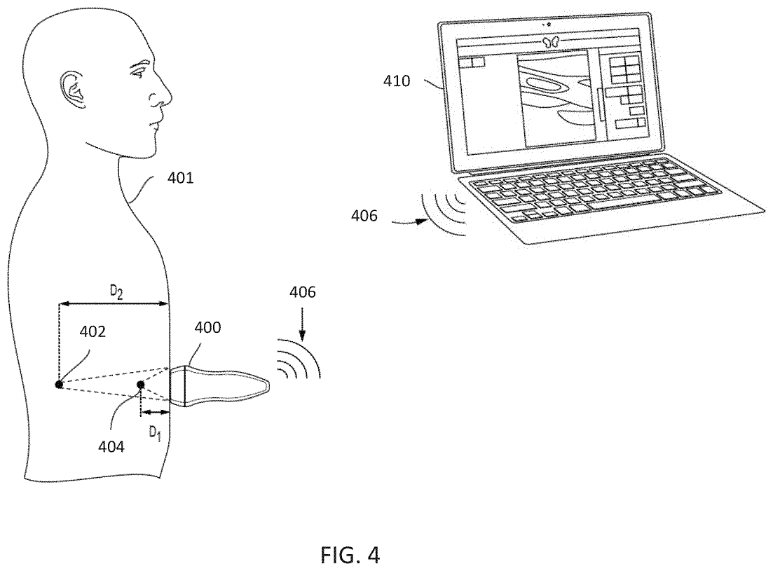

In some embodiments, the wireless communication circuitry includes one or more short- or long-range communication platforms. Exemplary short-range communication platforms include, ZigBee, Bluetooth (BT), Bluetooth Low Energy (BLE), Near-Field Communication (NFC). Long-range communication platforms include, Wi-Fi and Cellular. While not shown, the communication platform may include a front-end radio, an antenna and/or other processing circuitry configured to communicate radio signals to an external device (not shown, but discussed further in conjunction with the external device 304 in FIG. 3 and the external device 410 in FIG. 4). As explained herein, the radio signal may include ultrasound imaging information obtained by the probe 100. Ultrasound information may include signals and information defining an ultrasound image captured by the probe 100. Ultrasound information may also include control parameters communicated from the auxiliary device to the probe 100. The control parameters may dictate the scope of the ultrasound image to be obtained by probe 100.

In some exemplary embodiments, the built-in wireless communication circuitry transmits control messages, including control messages to set up a wireless connection, to maintain a wireless connection, and to tear down a wireless connection. For example, the wireless communication circuitry can transmit periodic beacon signals according to IEEE 802.11 and other prevailing standards. The beacon signal may include a BLE advertisement. Upon receipt of the beacon signal or the BLE advertisement, an auxiliary device (not shown) may respond to probe 100. That is, the response to the beacon signal may initiate a communication handshake between probe 100 and the auxiliary device.

FIG. 1B is a diagram illustrating a second exemplary embodiment of a handheld probe 150 comprising an ultrasound probe with a connected wireless communication module 152, in accordance with some embodiments of the technology described herein. In some embodiments, the wireless communication module includes circuitry (e.g., FPGAs, ASICs, processors, CPUs, wireless communication circuits, antennas, etc.) configured to provide wireless functionality to the handheld probe 150. In some embodiments, the wireless communication module 152 is implemented as a dongle connected to the handheld ultrasound probe 150. In some embodiments, the wireless communication module 152 can be configured to connect to the probe using an existing interface or set of interfaces. For example, if the ultrasound probe 150 includes a USB port, an Ethernet port, a serial port, and/or some other port, the wireless communication module 152 can connect to the probe 150 using the existing interface.

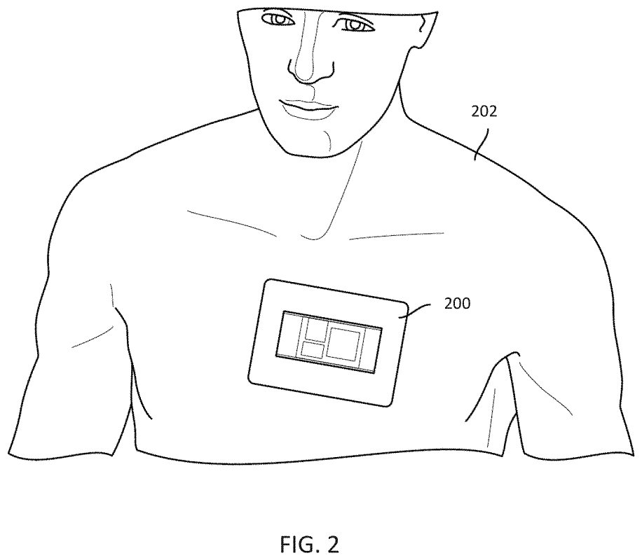

FIG. 2 illustrates an exemplary embodiment of patch 200 comprising an ultrasound probe and built-in wireless functionality, in accordance with some embodiments of the technology described herein. The patch 200 is coupled to patient 202. The patch 200 may be configured to wirelessly transmit data collected by the patch 200 to one or more external devices (not shown) for further processing. For purposes of illustration, a top housing of the patch 200 is depicted in a transparent manner to depict exemplary locations of various internal components of the patch. In some embodiments, the patch includes wireless communication circuitry that includes one or more short- or long-range communication platforms (e.g., BT, Wi-Fi, etc.). As explained herein, the wireless communication circuitry can be used to transmit ultrasound imaging information obtained by patch 200 (e.g., defining an ultrasound image captured by patch 200).

In some embodiments, an ultrasound probe may be embodied in a pill to be swallowed by a subject. As the pill travels through the subject, the ultrasound probe within the pill may image the subject and wirelessly transmit obtained data to one or more external devices for processing the data received from the pill and generating one or more images of the subject. For example, FIG. 3 illustrates an exemplary embodiment of a pill 300 comprising an ultrasound probe and wireless functionality, in accordance with some embodiments of the technology described herein. The pill 300 is configured to communicate wirelessly (e.g., via wireless link 302) with external device 304, which is configured to process data received from pill 300. A person may swallow pill 300 and, as pill 300 travels through the person's digestive system, pill 300 may image the person from within and transmit data obtained by the ultrasound probe within the pill to external device 304 for further processing. In some embodiments, the pill 300 may comprise an onboard memory and the pill 300 may store the data on the onboard memory such that the data may be recovered from the pill 300 once it has exited the person. Exemplary pill configurations are further described in U.S. Patent Application Publication No. 2017-0360397, assigned to the assignee of the present application, the contents of which are incorporated by reference herein in their entirety.

The external device 304 may be a desktop, a laptop, a smartphone, a handheld computing device, and/or any other device external to pill 300 configured for wireless communication. The external device may act as a gateway to cloud or internet communication and/or to other applications resident on the device. In an exemplary embodiment, the auxiliary device may include the patient's own smart device (e.g., smartphone) which communicatively couples to pill 300 and periodically receives ultrasound information from pill 300. The auxiliary device may then communicate the received ultrasound information to external sources.

In some embodiments, the auxiliary device may store ultrasound information received from pill 300. In another embodiment, the auxiliary device may relay ultrasound information received from pill 300 to another station. For example, the auxiliary device may use Wi-Fi to communicate the ultrasound information received from pill 300 to a cloud-based server, to a connected device, and/or the like. The cloud-based server may be a hospital server or a server accessible to the physician directing ultrasound imaging. The connected device may be a device connected to the auxiliary device via a wired and/or wireless connection. In another exemplary embodiment, pill 300 may send sufficient ultrasound information to the auxiliary device such that the auxiliary device may construct an ultrasound image therefrom. In this manner, communication bandwidth and power consumption may be minimized at pill 300.

In still another embodiment, the remote device may engage pill 300 through radio communication to actively direct operation of pill 300. For example, the remote device may direct pill 300 to produce ultrasound images of the patient at periodic intervals. The remote device may direct the depth of the ultrasound images taken by pill 300. In still another example, the remote device may control the manner of operation of the pill 300 (e.g., so as to preserve power consumption at battery). Upon receipt of ultrasound information from pill 300, the remote device may operate to cease imaging, increase the imaging rate and/or communicate an alarm to the patient or to a third party (e.g., physician or emergency personnel).

FIG. 4 is a diagram illustrating how a wireless ultrasound device may be used to image a subject, in accordance with some embodiments of the technology described herein. As shown in FIG. 4, ultrasound probe 400 is being used to image subject 401. The probe can operate in one or more modes, including modes of different frequencies. In the illustrative example shown in FIG. 4, when operating in a first mode (e.g., associated with a first frequency range, such as 1-3 MHz), the ultrasonic transducers in probe 400 may be configured to image the subject at or about a point 402, located at a depth D2 (e.g., 15-20 cm) from the subject's skin. When operating in a second mode (e.g., associated with a second frequency range, such as 6-8 MHz), the ultrasonic transducers in probe 400 may be configured to image the subject at or about a point 404, located at a depth D1 (e.g., 1-5 cm) from the subject's skin. While FIG. 4 shows operation at multiple modes, this is for exemplary purposes only and is not intended to be limiting.

The ultrasound probe 400 can be configured to wirelessly transmit data collected by the probe 400 to one or more external devices for further processing. For example, as shown in FIG. 4, ultrasound probe 400 may be configured to transmit data collected by probe 400 via a wireless connection (shown as 406) to the computing device 410 (a laptop in this non-limiting example), which may process the data to generate and display an image of the subject on a display.

In some embodiments, an ultrasound probe (e.g., probe 100, 150, 200 or 300) may be implemented in any of numerous physical configurations, and has the capabilities incorporated to perform imaging in modes as may be used when imaging with two or more of the following: a linear probe, a sector probe, a phased array probe, a curvilinear probe, a convex probe, and/or a 3D imaging probe. As another example, in some embodiments as described herein, the ultrasound probe may be embodied in a patch configured to be affixed to the subject (e.g., as shown in FIG. 2). As another example, in some embodiments, the ultrasound probe may be embodied in a pill (e.g., as shown in FIG. 3) to be swallowed by a subject and configured to image the subject as it is traveling through his/her digestive system. The aspects and embodiments described above, as well as additional aspects and embodiments, are described further below. These aspects and/or embodiments may be used individually, all together, or in any combination of two or more, as the technology described herein is not limited in this respect.

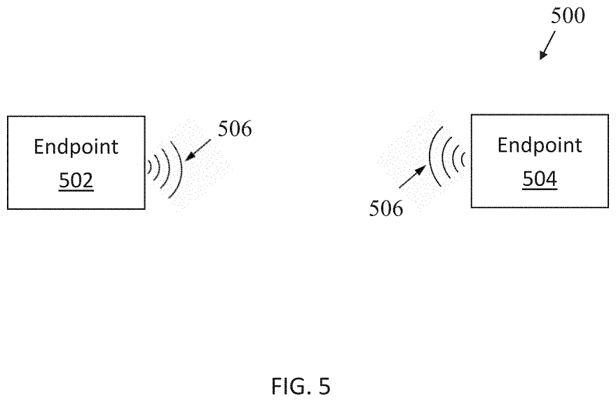

FIG. 5 illustrates a wireless architecture 500 with two endpoints 502 and 504, in accordance with some embodiments of the technology described herein. The endpoints 502 and 504 communicate via wireless communication link 506. In some embodiments, the endpoints 502 and/or 504 represent devices. For example, one of the endpoints 502 and/or 504 can be an ultrasound probe (e.g., probe 100, 150, 200 or 300), and the other endpoint can be a computing device (e.g., 304 or 410) to which the ultrasound probe is offloading ultrasound data, such as ultrasound image data. In some embodiments, the endpoints 502 and/or 504 represent logical components. For example, an endpoint can be used to logically refer to one or more ends of the wireless communication link 506 from a software and/or hardware perspective, as described further herein with respect to FIGS. 5-7.

In some embodiments, each endpoint can include a device, such as an ultrasound device and/or a computing device, with software, firmware, and/or hardware configured to communicate with and/or implement the wireless communication link. In some embodiments, the wireless communication link can be implemented using one or more classes (e.g., to abstract general components of the link from underlying hardware and/or implementation-specific details). For example, a link class can be used to implement the general link functionality, such as sending data, receiving data, and/or controlling the wireless communication link. By using one or more classes to abstract the link functionality from other aspects of the communication link (e.g., from the devices and/or the underlying medium, such as a wired or wireless communication link), the link functionality can be used to provide a standard interface for interacting with a link.

In some embodiments, the link class can provide an interface with a set of functions, including maintenance functions, functions to send and/or receive data, and other functions associated with the communication link. The link maintenance functions can include, for example, functions to create a link, to terminate or close a link, or to reset a link (e.g., if data is being dropped, if the connection is a poor connection, if data is being corrupted, etc.).

In some embodiments, the functions can be synchronous functions that expect a response. Synchronous functions can be used to send or receive data. For example, a send command can request the receiving endpoint to send data to the requesting endpoint (e.g., over a particular channel of the communication link) in response to the request. As another example, one function could write data to a memory location, and the response can indicate that the write succeeded (e.g., but does not send data back). As a further example, the function can request data to be directed to a particular memory location of the requesting device, and the requesting device expects to receive data in response to its request. Synchronous functions can be used for other purposes, such as to confirm delivery of a message, to request information, and/or to perform other functions for which a response is desirable.

In some embodiments, the functions are asynchronous functions that do not expect a responsive message. Functions used to send or receive data can be asynchronous functions. For example, a start function can be used by a requesting device to request data from a receiving device so that the requesting device can start receiving data from the receiving device. The start command can include arguments that specify related parameters, such as a buffer into which the data should be stored, the channel over which the requesting device wants to receive the data, and/or the like. The start function can, for example, configure the receiving device to stream data over a channel of the communication link into a buffer identified by the receiving device in its request. The requesting device can then read out the data from the buffer. Similarly, a device can send a stop function to terminate the read request. While such asynchronous functions may cause the receiving device to perform an action, the receiving device does not send a response to the transmitting device for the associated function (e.g., such that the requesting device does not wait for a response before performing further processing).

In some embodiments, the link class can be used to represent any type of communication link. For example, the link can be a wired communication link. A USB communication link, as an example, can include various dedicated hardware and/or software aspects, such as USB software libraries used to implement the USB protocol. The USB link may use different channels than other links (e.g., different channels than wireless links), and the data and/or protocol messages may be different than other links. The USB-specific libraries can be wrapped in a link class to abstract the underlying USB-specific implementation details to provide a standard interface that devices can use to communicate over any link. As another example, the link can be a wireless link. In some embodiments, a wireless link can be implemented using an IEEE 802.11 standard, such as one or more of IEEE 802.11 a/b/g/n/ac and/or the like. The wireless link can be used to offload ultrasound data from an ultrasound device using the wireless communication link at a data rate sufficient for offloading ultrasound data, such as a rate of 100 megabits/second, 150 megabits/second, 180 megabits/second, within a range of approximately 140-180 megabits/second, and/or the like. Thus, regardless of whether the link is a wireless link, a wired link (e.g., a USB link), and/or any other type of link as discussed herein, the link class can be used to provide a standard interface so that programs and/or devices can interface with any type of link. A device and/or computer program can be configured to use the link interface, which can allow different links to be used without needing to change the configuration of the device and/or computer program.

In some embodiments, one or more classes can be used to implement the device functionality. In some embodiments, a device class can be used to communicate over a link defined by one or more link classes. For example, a separate device class can be used to abstract the device functionality from the actual physical device(s) (e.g., from the hardware of an ultrasound device, a computing device, etc.). A device class can be implemented to utilize a standard link interface, which can allow the underlying type of link to change as discussed herein without requiring changes to the device class and/or the link class. In some embodiments, the device class can include code to detect a new link and to establish the communication link, including connecting the device to the link. In some embodiments, a device class can be an endpoint of a communication link, such as endpoints 502 and/or 504.

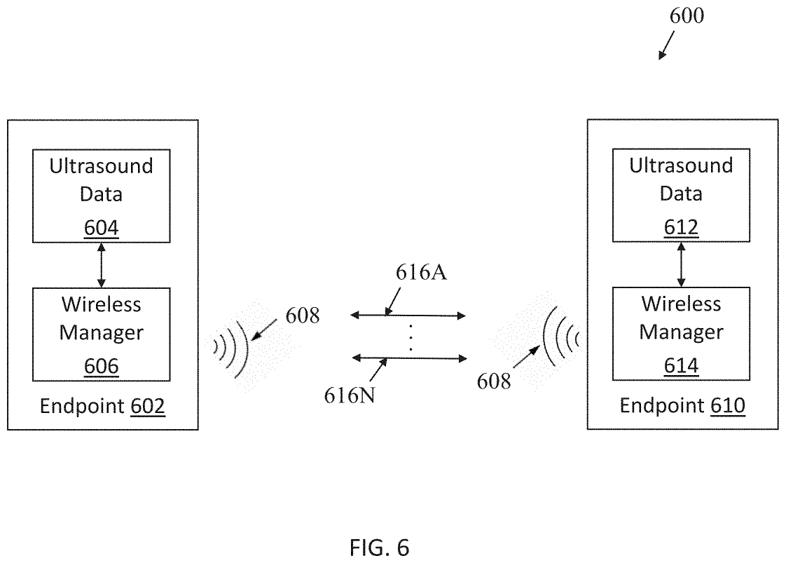

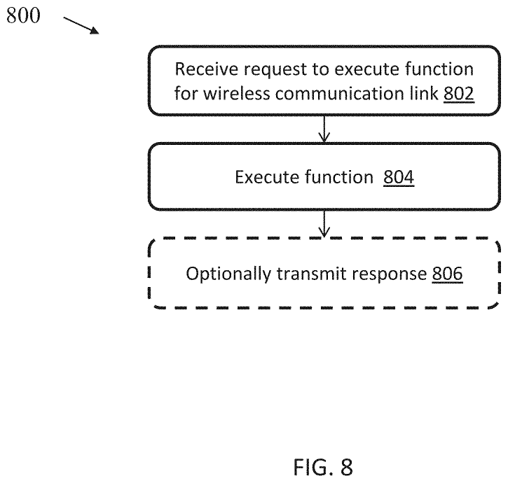

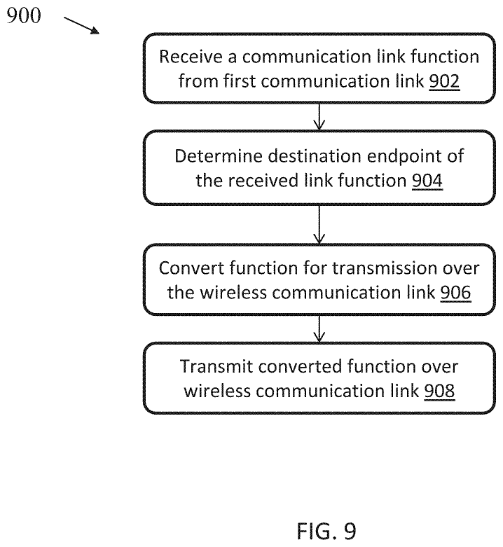

FIG. 6 illustrates a wireless architecture 600 with two endpoints 602 and 610, in accordance with some embodiments of the technology described herein. For example, the wireless endpoints 602 and 604 can be the endpoints 502 and 504 described in conjunction with FIG. 5. As shown in FIG. 6, each endpoint can include a wireless manager that manages the wireless communication link 608 between the endpoints 602 and 610. Endpoint 602 includes wireless manager 606, and endpoint 610 includes wireless manager 614. As described further in conjunction with FIGS. 8-9, the wireless managers 610, 614 can perform various functions for transmitting data to, and receiving data from, the wireless communication link 608. In some embodiments, the wireless managers 610, 614 obtain (e.g., receive, access, request, and/or the like) ultrasound data for transmission, and process the ultrasound data for the receiving device. For example, the wireless manager 606 obtains ultrasound data 604 for transmission across the wireless communication link 608, and performs the necessary processing of the ultrasound data 604 for transmission across the wireless communication link 608. Continuing with this example, the wireless manager 614 processes the received ultrasound data received by the endpoint 610 into the ultrasound data 612 (e.g., for processing by the device associated with endpoint 610).

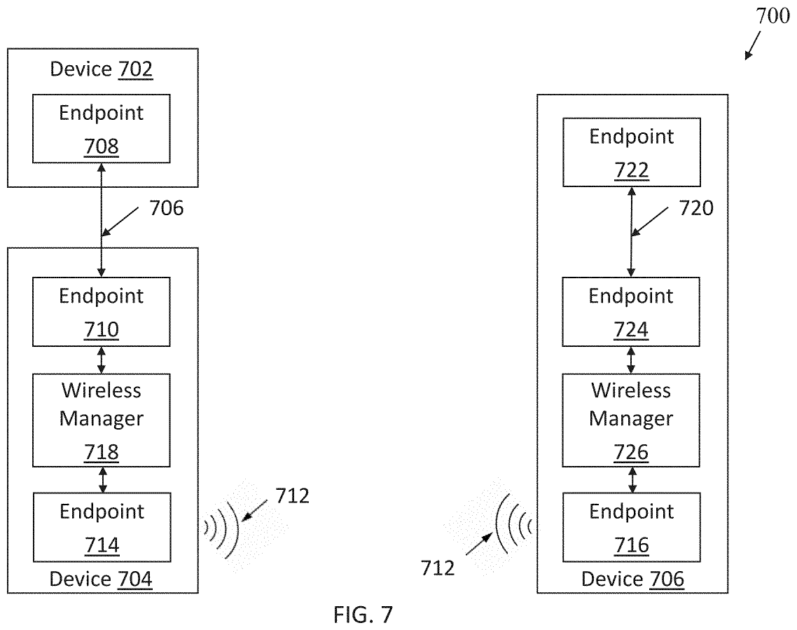

In some embodiments, the wireless manager 606, 614 can provide forwarding functionality to forward data and/or messages from one communication link over another communication link, as discussed further in conjunction with FIGS. 7 and 9. While FIG. 6 shows the wireless managers as components of the endpoints, this is for illustrative purposes only. For example, the functionality of the wireless managers can be implemented as part of an endpoint, the wireless manager may provide the endpoint to a communication link (e.g., such that there is no separate endpoint), and/or a device can implement both the endpoint and wireless manager (e.g., as part of a device class).

The wireless communication link 608 can include one or more channels, shown as 616A through 616N, collectively referred to as channels 616. For example, one or more channels 616 can be used to exchange data (e.g., ultrasound data), one or more channels 616 can be used for transmitting control messages (e.g., messages used to create and/or maintain the wireless communication link, function calls, etc.), and/or the like. In some embodiments, if the wireless communication link is implemented using a link class as described herein, the link class can include one or more channels that are used to transmit various types of data, such as ultrasound data and/or control data. Each type of wired and/or wireless communication link may use different channels, which can be abstracted out using the link class (e.g., such that the device need not know about the various channels, which are handled by the link class). The wireless managers 606, 614 can be configured to set up and use the channels 616 to communicate data, as will be explained further in conjunction with FIGS. 8-9.

As an illustrative example, a communication link can be configured with send and receive channels, control channels, and/or the like. For example, a USB communication link can be implemented with two channels, one to send data (e.g., including ultrasound data, control data, etc.) and one to receive data. As another example, a wireless communication channel can include various channels (e.g., ports), where different ports are used to send control data, receive control data, to send ultrasound data, and/or to receive ultrasound data.

FIG. 7 illustrates a wireless architecture 700 with four communication links, in accordance with some embodiments of the technology described herein. The example shown in FIG. 7 includes three devices 702, 704 and 706. Device 702 is in communication with device 704 via communication link 706 (e.g., a wired or wireless communication link) that is formed by endpoints 708 and 710, with endpoint 708 associated with device 702, and endpoint 710 associated with device 704. Device 704 is in communication with device 706 over wireless communication link 712 that is formed by endpoints 714 and 716, with endpoint 714 associated with device 704 and endpoint 716 associated with device 706. Device 704 includes wireless manager 718 that controls the transmission of data between endpoints 710 and 714. Device 706 also includes a communication link 720 that is formed by endpoints 722 and 724. Device 706 includes wireless manager 726 that controls the transmission of data between endpoints 716 and 724.

In some embodiments, the wireless managers 718, 726 can be used to provide the wireless link 712 in a manner that is transparent to the endpoints 708 and/or 722. For example, the endpoint 708 can be configured to communicate over the communication link 706 such that endpoint 708 only needs to be aware of the communication link 706 (e.g., and not the communication links 712 or 720). Similarly, the endpoint 722 can be configured to communicate over the communication link 722 such that endpoint 722 only needs to be aware of the communication link 720 (e.g., and not the communication links 706 or 712). Thus, one or more wireless managers can be configured to add an additional communication link without requiring adjustments to existing configurations. In this example, the wireless managers 718, 726 can add the wireless communication link 712 in a manner that does not require changes to endpoints 708 (e.g., including the device 702) and/or the endpoint 722. As discussed herein, remote function calls can be used to provide the wireless communication link 712 in a manner that is transparent to both endpoints 708 and 722.

In some embodiments, the wireless managers and/or one or more additional communication links can be added using software and/or hardware. For example, device 704 can be an additional device connected to device 702 to add wireless transmission functionality to device 702 so that data from device 702 can be transmitted over the wireless communication link 712. For example, referring to FIG. 1A, the ultrasound probe 100 can include both the device 702 and the device 704, where the device 704 represents additional hardware and/or software incorporated into the probe 100 to facilitate wireless communication.

As another example, referring to FIG. 1B, the device 702 can be the ultrasound probe 150, and the device 704 can be the wireless communication module 152. The wireless communication module 152 can be configured to connect to the ultrasound probe 150 using the type of communication link supported by the ultrasound probe 150 (e.g., so that the software and/or hardware of the ultrasound probe 150 need not be changed in order to add wireless functionality to the ultrasound probe 150). For example, if the device 702 is configured to use a type of wired connection (e.g., USB), the device 704 can be configured to connect to the device 702 using its existing wired connection. In some embodiments, the device 704 can be a dongle that includes a physical interface for the communication link 706, as well as necessary hardware (e.g., a processor) and software to provide endpoint 710 for the communication link 706 and the wireless manager 718 to convert data from the communication link 706 to the communication link 712.

As a further example, device 706 can include software (e.g., including wireless hardware, if necessary) to add wireless support to the device 706. For example, the device 706 can be a laptop, smartphone, or mobile phone. The device 706 can include necessary software to provide the endpoint 716 for the wireless communication link 712, the wireless manager 726, and the endpoint 724. The software and/or hardware added to device 706 to support the wireless communication link 712 can avoid needing to change aspects of the existing configuration of the device 706, such as the endpoint 722 and/or the program(s) configured to use the communication link 720. For example, device 706 can be a smartphone, which includes an original computer program configured to receive the ultrasound data from the ultrasound device using the communication channel 720 via endpoint 722. The smartphone can include a new computer program configured to implement one or more of the endpoint 716, the wireless manager 726, and the endpoint 724. The new computer program can therefore receive the ultrasound data from the wireless communication link 712 and provide it to the original computer program (e.g., via endpoint 722) using the communication link 720 that the smartphone does not need to be physically connected to the ultrasound device.

In some embodiments, one of the wireless managers 718, 726 can act as a server, and the other wireless manager (and/or additional wireless managers) can be clients. For example, the server can run on the ultrasound device (and/or device(s) connected to the ultrasound device, such as a wireless communications module), and the client can run on each of the computing devices or phones that connects to the ultrasound device. In some embodiments, the data transfer can be client driven (e.g., by client endpoint(s) connected to a server endpoint). For example, a client can request data from the server, and the server can transmit the data in response to the request. As another example, the client can request a connection to an associated endpoint to receive data from the endpoint as it becomes available via a streaming data request, and the server can transmit the data accordingly (e.g., such as sending it on a particular channel and/or destined for a particular buffer associated with the streaming request).

In some embodiments, multiple devices can connect to an ultrasound device to receive ultrasound data and/or interact with the ultrasound device. For example, the server version of the wireless manager can be configured to manage a plurality of wireless connections with client devices. In some embodiments, the server can keep track of which functions are called, including by which devices. In some embodiments, the server may be associated with and/or connected to a plurality of ultrasound devices (e.g., with a plurality of ultrasound probes). In some embodiments, the server can be configured to associate each ultrasound device and computing device with an associated identifier (e.g., a unique identifier) in order to manage communication links and/or messages communicated between various ultrasound devices and other devices over those links. The server can be configured to set up and maintain connections between the various devices.

Each device can register with the server in order to connect to another device (e.g., to connect to an ultrasound device associated with the server). For example, a new device can register with the server, and the server can coordinate the necessary messages to inform a desired destination device of the new device, and to create a connection between the new device and the desired device. For example, the server can message the desired device to indicate the new device (e.g., a new ultrasound probe) is requesting a connection. The desired device can accept a connection request from the new device, and the server can create a new mapping between the new device and the desired device for the new communication link. Once the mapping is created, the server is configured to route messages between the new device and the desired device over the communication link.

The number of devices, endpoints, and wireless managers shown in FIG. 7 is for exemplary purposes and is not intended to be limiting. For example, a device can be directly associated with one or more communication links, including both wired and/or wireless communication links, such that the device serves as the endpoint. As another example, the wireless manager can serve as one or more endpoints (e.g., such that the wireless manager 718 implements the endpoint 710 and/or the endpoint 714). Each device may include a wireless manager to manage communications across the wireless link, and/or the wireless manager functionality can be incorporated into other aspects of the device (e.g., communication layers, communication hardware, and/or the like). In some embodiments, a device may include one or more internal communication links. For example, a device can join different types of communication links using a wireless manager. A device could, for example, communicate with one or more devices via a wireless protocol, and the wireless manager could convert the received data for transmission over a different communication link for further processing. For example, if the device is configured to process data using a particular protocol, such as Bluetooth, USB, WiFi, ZigBee, NFC, and/or some other protocol, the device can still communicate with devices via a link using a different protocol using a wireless manager to essentially bridge the two links.

As described further herein, in some embodiments, the wireless manager 720 can be configured to perform a translation from one communication link using a first protocol to another communication link using a different communication protocol. For example, if the communication link 706 leverages a different communication protocol than wireless communication link 712, the wireless manager 718 can be configured to essentially serve as an intermediate endpoint to both communication links, such that data transmitted over communication link 706 can be transmitted over communication link 712 and vice versus.

In some embodiments, the communication link, such as the wireless communication link 712, supports remote function calls, such as remote procedure call (RPC) calls, to pass communication link functions across multiple communication channels. For example, referring to FIG. 7, endpoint 722 can invoke one or more functions associated with the communication link 720, and the wireless manager 726 can make one or more associated remote function calls across the wireless communication link 712 to cause the endpoint 710 to send the one or more functions invoked by endpoint 722 to the endpoint 708 over the communication link 706. Similarly, wireless manager 718 can use one or more remote function calls to cause the endpoint 722 to receive a function call over link 720 from endpoint 724 that was invoked by the endpoint 708 over the communication link 706. Therefore, the wireless managers can make calls to other communication links using the remote function calls. The wireless managers 718, 726 can leverage the remote function calls in order to pass synchronous and asynchronous functions from endpoint 708 to endpoint 722, and vice versus.

FIG. 8 is a flowchart of an illustrative process 800 for operating a wireless communication link, in accordance with some embodiments of the technology described herein. Illustrative process 800 may be performed by any suitable device(s) and, for example, may be performed by an ultrasound device and/or a computing device in connection with an ultrasound device, and/or components of such devices, such as a wireless manager and/or endpoint of the device.

At step 802, the device receives data associated with a request to execute a remote function call for a wireless communication link. As described herein, the device can be an ultrasound device, such as an ultrasound probe, pill, patch, wireless device connected to connected to an ultrasound device, and/or other ultrasound device configured to provide a wireless communication link as described herein. As also described herein, the wireless communication link can include two endpoints and/or devices, one on each end of the communication link. The device can be one of the endpoints, and the device can receive the request from a different communication link.

In some embodiments, the device can include a wireless manager configured to receive the request of step 802. For example, referring to FIG. 7 the wireless manager 718 can receive the request of step 802 via the wireless communication link 712. The request can be generated by the wireless manager 726 to pass a message sent from the endpoint 722 via the communication link 720. For example, the endpoint 722 may call one or more functions associated with the communication link 720 destined for endpoint 708 (e.g., without knowledge of the addition of the wireless communication link 712 between endpoints 722 and 708). The wireless manager 726 can send one or more remote function calls at step 802 to the wireless manager 718 to cause the wireless manager 718 to execute the one or more functions over communication link 706 to endpoint 708.

At step 804, the device executes a function associated with the request received at step 802. As described herein, remote function calls, such as those provided by a RPC protocol, can be used so that functions can be called and invoked remotely, such that the device can make function calls on the communication link. The functions can include link maintenance functions, functions to send data, functions to receive data, and/or the like, as described herein. For example, referring to FIG. 7, the wireless manager 718 of device 704 can execute one or more functions to make function calls to endpoint 708 over communication link 706 using the link interface for the communication link 706. As an illustrative example, device 706 (e.g., via endpoint 722) may request to receive ultrasound data from device 702, which is an ultrasound device configured to generate data. The endpoint 722 can generate the associated message(s) to request the ultrasound data from device 702. The wireless manager 726 passes the associated message(s), which include the requisite function calls from the link interface for communication link 720, over the wireless link 712 to wireless manager (e.g., which the wireless manager receives at step 802). The wireless manager 718 can execute a function that essentially passes the associated message(s) to the device 702 via the communication link 706 by making the same calls to the link interface for communication link 706 that the endpoint 722 made for communication link 720.

At step 806, the device optionally transmits a response to the device or endpoint of the wireless communication link from which it received the request at step 802. As described herein, a synchronous function call for a communications link interface can be configured to receive a response from the receiving endpoint. In some embodiments, the endpoint that transmits the asynchronous function call can be configured to wait for a response until proceeding with its further execution. Therefore, in some examples, the response can be used to coordinate and/or control the execution of the requesting device (e.g., in the event the function was not executed, did not execute properly, etc.). Continuing with the example for step 804, if the endpoint 722 sends a synchronous function call to endpoint 708, then the endpoint 722 can be configured to wait for a response. When processing the remote function call that the wireless manager 726 made to pass the function call over the wireless endpoint 712, the wireless manager 718 can be configured to make the synchronous function call to endpoint 708 over the communication link 706, and to wait for a response from the endpoint 708. The wireless manager 718, upon receipt of a response from the endpoint 708, can make one or more remote function calls to the wireless manager 726 over the wireless communication link 712 to cause the wireless manager 726 to send the response to the endpoint 722 over the communication link 720.

As also described herein, since some functions are asynchronous and therefore do not require a response, this step is optional. For example, an asynchronous read request can configure the requesting device to constantly read data from a buffer (or data stream) when the data arrives, and not in response to any further function call or response from the device transmitting the data. In some embodiments, one or more channels of a communication link can be used and/or configured for streaming data (e.g., imaging data). As an illustrative example, the techniques described herein can allow the endpoint 722 to send an asynchronous read request to the endpoint 708. Upon receipt of an asynchronous read request from the endpoint 722 over the communication link 720, the wireless manager 726 can invoke the necessary remote function call(s) to cause the wireless manager 718 to send the asynchronous read request to the endpoint 708 via the endpoint 710 over communication link 706. The asynchronous read request can cause the endpoint 708 to begin to stream data to the endpoint 722 as it becomes available (e.g., ultrasound data). When the wireless manager 718 receives streaming data from the endpoint 708 destined for the endpoint 722, the wireless manager 718 can process the streaming data so that it is forwarded to the endpoint 722 (e.g., so that the streaming data is put into a streaming receive buffer at the endpoint 722). In some embodiments, the wireless manager 718 can include additional data along with the streaming data to indicate to the wireless manager 726 that the data is streaming data. The wireless manager 726 can be configured to process the additional data, and to use such data to determine how to transmit the data to the endpoint 722 (e.g., over a particular channel in an asynchronous manner). The device 706 can include a program that monitors the receive buffer so that it processes received data. Such asynchronous streaming configurations can be advantageous for streaming applications such as ultrasound streaming, because each piece of image data need not be requested and/or acknowledged, which can reduce the overhead required to otherwise transmit ultrasound data over one or more communication links.

As described herein, the techniques provide architectures for implementing communication links for ultrasound devices. As also described herein, different communication links can be implemented differently (e.g., using different channels, configurations, interfaces, and/or the like). For example, while one communication link uses certain channels to transmit data and control messages, a different communication link may use an entirely different set of channels to transmit the same data and control messages. Therefore, the wireless managers can be configured to translate from one communication link to another communication link (e.g., across channels, message types, and/or the like). FIG. 9 is a flowchart of an illustrative process 900 of converting data from one communication link for transmission over a wireless communication link, in accordance with some embodiments of the technology described herein. Illustrative process 900 may be performed by any suitable device(s) and, for example, may be performed by an ultrasound device and/or a computing device in connection with an ultrasound device.

At step 902, a device (e.g., a wireless manager) receives a communication link function from a first communication link. The device can be, and/or be associated with, an endpoint of the first communication link. The communication link function can be a call to a function of the communication link interface, streaming ultrasound data, and/or other data and/or messages transmitted over the first communication link.

At step 904, the device determines the destination for the received communication link function. As described herein, a server implementation of the wireless manager can be configured to store association information for each connected client endpoint and/or communication link. The server can, for example, create and or build a database for each connection, including the associated endpoints of the connection, a status of the connection (e.g., whether there is a pending synchronous function called that expects a response), and/or the like. The server can use such a database to determine how to process data and/or messages received from the endpoints. In some embodiments, each wireless manager can keep track of the various functions called for a particular link, including associated arguments, etc. For example, if a wireless manager receives a remote function call for execution, the wireless manager can keep track of the function call, whether any response has been provided, etc. The wireless manager can use such data to determine how to route information received from endpoints (e.g., if there is a pending function call waiting for a response and the wireless manage receives the response, it will return the response for that function call).

At step 906, the device converts the communication link function received at step 902 from the first communication link for transmission over the wireless communication link. For example, referring to FIG. 7, the wireless manager 718 can perform the conversion from the communication link 706 to the wireless communication link 712. In some embodiments, the conversion requires mapping channels from the first communication link to channels of the wireless communication link. For example, if the first communication link is a USB communication link, the device can perform a mapping from the USB channels to the channels used for the wireless communication link (e.g., channels used by the TCP link). For example, if the device receives data on channel 1 via a USB link, the device can transmit the same data on the associated channel of the wireless link, such as channel 6.

In some embodiments, the conversion process can include generating additional information. For example, the server can encode additional data prior to transmitting the data over the next communication link (e.g., so that other wireless managers in the communication chain can appropriately determine how to process the data, such as determining the destination of the data, on which channel(s) the data should be sent, and/or the like). For example, each function (e.g., of a communication link interface) can include an associated function name, data (e.g., function arguments, ultrasound data, etc.), type (e.g., asynchronous/synchronous), channel over which the function should be sent, and/or the like. When converting a communication link function for transmission over the wireless communication link, the device can generate additional information to indicate such associated information. For example, to encode a function call to a communication link function as a remote function call, the device can generate sufficient data to make a remote function call to the link interface function with the associated data and arguments. If the function is synchronous and therefore expects a return value, the device can be configured to wait for that return value.

In some embodiments, the device can generate a packet for the wireless communication link. For example, the body of the packet can include the original message and/or data received from the first communication link, and the header can include information used to route the packet across the wireless communication link and/or across additional links in the path to the ultimate destination. For example, if the device receives streaming ultrasound data on channel 1 via the USB link, the device can encode the received data into a packet that has a header specifying the data is for channel 1 of a USB link. When the new packet is received, the receiving device can de-multiplex the packet from the wireless communication link to the appropriate channel 1 of a USB link to the ultimate receiving endpoint.

The receiving wireless manager can be similarly configured to encode data routed over the wireless communication link. For example, if the receiving wireless manager (e.g., on an ultrasound device) receives a packet from the wireless communication link that includes a remote function call, the wireless manager can decode the packet to determine a communication link function call and its appropriate arguments, and call the function (e.g., to make a call on another communication link, as described herein). For example, referring to FIG. 7, a device (e.g., device 706, such as a smartphone) can include hardware and/or software configured to encode a function call that is made by endpoint 722 for communication link 720 into a new data packet to make a remote function call, and send the new data packet over the wireless communication link 712 to an ultrasound device implementing devices 702 and 704. The ultrasound device, including via the wireless manager 718, can execute the request encoded in the packet, and ultimately generate a new packet with the response information (e.g., to indicate it is a response to a particular call to a particular function). The ultrasound device can transmit the response packet to the device 706, which the wireless manager 726 can process to provide the response to the endpoint 722.

In some embodiments, aspects of devices and/or communication links can be modified to support adding a wireless communication link. For example, some wired links may be able to transmit data at a frame rate that is faster than a wireless link. Sending too much data too quickly could overload endpoints of the wireless link. The system can be adjusted to take into account use of a wireless communication link, such as by modifying parameters of the ultrasound device and/or wireless link so that the devices and/or other communication links transmit data in a manner compatible with the wireless link. In some embodiments, one or more parameters of the ultrasound device can be modified to increase and/or decrease the amount of ultrasound image data captured, the speed at which the data is captured, and/or the like. For example, the ultrasound device can include a pulse repetition interval parameter that specifies how quickly to send and receive data. This parameter and/or other parameters can be adjusted appropriately to adapt the amount and/or speed of data generated from the ultrasound device to take into account use of a wireless communication link.

At step 908, the device transmits the converted communication link function over the wireless communication link. For example, the ultrasound device (e.g., probe, pill, or patch) can wirelessly offload converted ultrasound data to a remote computing device, as shown and discussed in conjunction with FIGS. 2-4. As another example, the ultrasound data can be wirelessly offloaded from a second device physically connected to an ultrasound device, such as the wireless communication module 152 connected to the probe 150 as shown in FIG. 1B.

In some embodiments, devices in communication over a plurality of communication links (e.g., such as devices that are not the outer-most endpoints of the communication path made up of multiple communication links) can use techniques coordinate with each other to adjust the amount of data being sent between the devices. For example, if two devices in communication over a wired communication link and one device is streaming data to a receiving device, the receiving device can send commands to the streaming device to control the amount of data sent (e.g., to reduce the amount of data being sent, the rate at which the data is sent, and/or to not send data for a period of time). Otherwise, if the receiving device cannot keep up with processing the data being streamed, various receive buffers and/or transmit buffers can back up and/or overflow. In some embodiments, the techniques can be used to reduce the frame rate at which the ultrasound data captures ultrasound images, which can in turn reduce the amount of data being streamed to the device.

For example, referring to FIG. 7, ultrasound data for transmission from the device 702 to the device 706 could be buffered at the device 702 (e.g., at endpoint 708), the device 704 (e.g., at endpoints 710, 714 and/or the wireless manage 718), and/or at the device 706 (e.g., at endpoints 716, 724 and/or wireless manager 726). The type of communication link may cause one or more devices to buffer data more than others, such as devices on the sending side of the wireless communication link 712. The techniques can provide mechanisms for each of the various endpoints, devices, and/or wireless managers in the communication path to reduce the amount of data sent from device 702. For example, while a higher data rate could be handled by a wired communication link 706, a lower data rate may be supported by the wireless communication link 712. The device 704, such as the wireless manager 718, could therefore end up buffering more data as it queues for transmission over the wireless communication link 712. The techniques can allow for the wireless manager 718 to control the amount of data being sent by the device 702, even though the wireless manager 718 is not the destination of the ultrasound data.

In some embodiments, the bandwidth of a communication link may vary over time. For example, due to various types of interference, interference sources, and/or the number of devices using available spectrum, the bandwidth of a particular wireless communication link may change over time. The techniques can provide for dynamic control of data transmission based on current communication link conditions. The techniques can be configured to send data at the highest rate supported by all communication links, and to adjust that rate as link conditions change over time.

The ultrasound devices described herein may be used for a broad range of medical imaging tasks including, but not limited to, imaging a patient's liver, kidney, heart, bladder, thyroid, carotid artery, lower venous extremity, and performing central line placement. In particular, the ultrasound device may be configured to wirelessly offload ultrasound data sufficient to construct an ultrasound image containing information indicative of these indications. In some embodiments, the ultrasound device may be used to monitor tumor size and/or shape, tumor elasticity, and/or tumor vascularity. In some embodiments, the ultrasound device may be used to monitor bladder fullness and/or volume, and/or bladder outlet obstruction. In some embodiments, the ultrasound device may be used to monitor pulse wave velocity and/or blood pressure.

In some embodiments, the ultrasound device may be used to monitor inferior vena cava (IVC) diameter, IVC collapsibility, IVC blood flow rate, and/or IVC wall motion. In some embodiments, the ultrasound device may be used to monitor intravascular volume, left ventricular wall motion, cardiac output, ejection fraction, central venous pressure, and/or blood flow rate. In some embodiments, the ultrasound device may be used to monitor for abdominal aortic aneurysm, to monitor for deep vein thrombosis, to monitor fetal heartbeat, to monitor for pregnancy, and/or to monitor follicle growth in ovaries. In some embodiments, the ultrasound device may be used to monitor for dehydration using ultrasound imaging of the IVC, to monitor skin moisturization, to monitor amniotic fluid volume, and/or to monitor for pneumonia.

In some embodiments, the ultrasound device may be used to monitor for pleural effusion, to monitor for pneumothorax, to monitor for chronic obstructive pulmonary disease (COPD), and/or to monitor lung volume. In some embodiments, the ultrasound device may be used to monitor enteral feeding tube placement, to monitor gastric emptying, to monitor for indigestion, to monitor kidney function, to monitor renal blood flow rate, to monitor needle placement during pericardiocentesis, to monitor needle placement during amniocentesis, to monitor for vasospasm, to monitor high-intensity focused ultrasound ablation, to monitor drug delivery, and/or to monitor hemodialysis access.

In some embodiments, the ultrasound device may be used to monitor the vascular system, the liver, the gallbladder, the biliary system, the spleen, the pancreas, the gastrointestinal tract, the peritoneal cavity and abdominal wall, the urinary system, the retroperitoneum, the breast, the thyroid and parathyroid glands, the scrotum, the musculoskeletal system, the neonatal and pediatric abdomen, the neonatal and pediatric adrenal and urinary system, the neonatal and infant head, the infant and pediatric hip, the neonatal and infant spine, the thoracic cavity, the cerebrovascular system, the female pelvis, the uterus, the ovaries, the adnexa, obstetric measurements, gestational age, fetal growth, fetal congenital abnormalities, the placenta, the umbilical cord, amniotic fluid, fetal membranes, fetal hydrops, the fetal face and neck, the fetal neural axis, the fetal thorax, the fetal anterior abdominal wall, the fetal abdomen, the fetal urogenital system, the fetal skeleton, fetal situs, and fetal nuchal translucency.

As also described herein, one or more communication links can be used to join other communication links. For example, additional and/or new communication links can be added to existing system configurations without needing to modify some of the existing hardware and/or software components. In some embodiments, for example, a new type of communication link can be added to a system by modifying only the necessary hardware and/or software needed to interface with the existing communication links of the system.

As described herein, the techniques can include implementing classes to abstract functionality for communication links and devices away from the implementation-specific details of the system (e.g., a link class, which has an interface with standard commands such as send, receive, etc.). If a link is being used to join endpoints together that are communicating over one or more additional links, then the link can be designed to use remote procedure calls that can pass function calls from one communication link across a second (or more) additional communication link(s). Therefore, links can be implemented with remote function calls designed to allow functions called in one link to be passed across other communication links to the ultimate destination endpoint (e.g., functions called via an existing USB link can be passed across a second link, such as a wireless TCP link, using remote function calls in the wireless link). Such links with remote function calls can be used to add additional links in a transparent manner, such that the endpoint still thinks it is communicating with a particular link implementation, but the new link is forwarding calls from that endpoint to another device over one or more additional links the endpoint is not, and need not be, aware of.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

What is claimed is:

1. A method, comprising:
implementing a wireless communication link for ultrasound data using a set of functions to wirelessly transmit the ultrasound data.

2. The method of claim 1, wherein one or more functions of the set of functions can be executed using a remote procedure call (RPC) protocol.

3. The method of claim 1, further comprising receiving, from an endpoint of the wireless communication link, a request to execute a function from the set of functions.

4. The method of claim 3, further comprising executing a function associated with the request to execute the function from the set of functions.

5. The method of claim 3, further comprising transmitting a response to the endpoint.

6. The method of claim 1, wherein a function from the set of functions comprises an asynchronous function.

7. The method of claim 1, wherein a function from the set of functions comprises a synchronous function.

8. The method of claim 1, wherein a function from the set of functions comprises a reset function.

9. The method of claim 1, wherein a function from the set of functions comprises a send function.

10. The method of claim 1, wherein a function from the set of functions comprises a receive function.

11. The method of claim 1, wherein a function from the set of functions comprises a start function.

12. The method of claim 1, wherein a function from the set of functions comprises a stop function.

13. The method of claim 1, wherein the wireless communication link comprises a set of channels.

14. The method of claim 13, wherein the set of channels comprises a transmit channel and a receive channel.

15. The method of claim 1, further comprising:
receiving ultrasound data from a wired link; and
transmitting, via the wireless communication link, at least a portion of the ultrasound data to an endpoint of the wireless communication link.

16. The method of claim 15, wherein:
the wired link comprises a set of channels; and
transmitting comprises transmitting data from a first channel of the set of channels of the wired link over a second channel of the set of channels of the wireless communication link.

17. The method of claim 16, wherein the first channel is different than the second channel.

18. The method of claim 1, further comprising:
receiving data from the wireless communications link; and
transmitting, via a second link, at least a portion of the data to an endpoint of the second link.

19. The method of claim 18, wherein:
the second link comprises a set of channels; and
transmitting comprises transmitting data from a first channel of the set of channels of the wireless communication link over a second channel of the set of channels of the second link.

20. The method of claim 1, wherein the ultrasound data is sufficient for forming one or more ultrasound images therefrom.
